(19) United States
(12) Patent Application Publication
(10) Pub. No.: US 2024/0368631 A1
(43) Pub. Date: Nov. 7, 2024

(54) Genetically Engineered Eukaryotic Cells and Cell Lines and Uses Thereof

(71) Applicant: Andrew E. Syvyk, Bryan, TX (US)

(72) Inventor: Andrew E. Syvyk, Bryan, TX (US)

(73) Assignee: The Texas A&M University System, College Station, TX (US)

(21) Appl. No.: 18/650,593

(22) Filed: Apr. 30, 2024

Related U.S. Application Data
(60) Provisional application No. 63/499,281, filed on May 1, 2023.

(51) Int. Cl. C12N 15/90; C07K 14/005; C12N 5/071; C12N 9/12

(52) U.S. Cl. CPC C12N 15/907; C07K 14/005; C12N 5/0686; C12N 9/1241; C12N 2510/00; C12N 2750/14122; C12N 2750/14152; C12N 2800/30; C12N 2800/90; C12N 2830/002

(57) ABSTRACT

Provided herein are stably-modified cell lines such as HEK293 cell lines. The cell lines are modified with an insertion, such as, a transposon containing an inducible recombinase and a promoter and one of a DNA cassette encoding an excisable mitogen, a DNA cassette encoding an inducible gene of interest or a DNA cassette encoding the excisable mitogen and the inducible gene of interest. The stably-modified cell lines are used in methods for producing a biological medical product, for example, in a scalable process for producing biopharmaceuticals.

### CROSS-REFERENCE TO RELATED APPLICATIONS

This non-provisional patent application claims benefit of priority under 35 U.S.C. § 119(e) of provisional application U.S. Ser. No. 63/499,281, filed May 1, 2023, the entirety of which is incorporated by reference.

### BACKGROUND OF THE INVENTION

### Field of the Invention

The present invention relates to the fields of genetic engineering of cells and protein production therefrom. More particularly, the present invention relates to human cells engineered to switch from a normal or overexpressed mitotic cycle to expression of a difficult to express protein or toxic protein.

### Description of the Related Art

The demand for biopharmaceuticals and cost effective scalable bioproduction thereof has been increasing. Mammalian cell lines, such as HEK293 cell lines are used for biomanufacturing recombinant protein and viral vectors. An inducible system based on DNA recombination, however, will not be able to generate recombination events in all treated cells. Moreover, many proteins of commercial value are toxic to the cells or significantly inhibit cell proliferation that limits scaling up of cell culture and makes production inefficient and expensive.

Thus, there is a recognized need in the art for engineered cell lines and methods enabling the production of difficult proteins therefrom. Particularly, the prior art is deficient in engineered human cells that switch from enhanced mitotic cycling to production of at least one protein including difficult to express proteins to scale-up production thereof. The present invention fulfills this longstanding need and desire in the art.

### SUMMARY OF THE INVENTION

The present invention is directed to a stably-modified cell line. The cell line comprises an insertion containing an inducible recombinase and a promoter. The present invention is directed to a related stably-modified cell line further comprising a DNA cassette encoding an excisable mitogen, a DNA cassette encoding at least one inducible gene of interest (GOI) or a DNA cassette encoding the excisable mitogen and the at least inducible gene of interest.

The present invention is further directed to a method for producing a biological medical product. In this method, expression or overexpression of the gene encoding the mitogen is induced in a population of cells comprising the stably-modified cell line described herein and cell number in the population is increased via activity of the expressed or overexpressed mitogen. A recombination activity of the inducible recombinase comprising the transposon in the cells in the increased cell population is induced. Simultaneously the expression or the overexpression of the mitogen is switched off and expression of the inducible gene of interest comprising the cells in the increased cell population is induced to produce a protein or a viral particle proteins of medical or therapeutic interest encoded by the at least one inducible gene of interest. The protein or the viral particle protein is isolated from the cell population, thereby producing the biological medical product.

The present invention is directed further to a scalable process for producing at least one biopharmaceutical destructive to a population of host cells stably modified to express the same. In this process, a ratio of the host cells induced to express the biopharmaceutical to the host cells in a normal mitotic cycle or an enhanced mitotic cycle is adjusted at least once in the population.

The present invention is directed further still to an engineered HEK293 cell line. The HEK293 engineered cell line comprises an insertion containing an inducible recombinase and a promoter and a DNA cassette encoding an excisable mitogen and at least one inducible gene of interest.

Other and further aspects, features, benefits, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIGS. 1A-1C illustrate the parental vector design and functional analysis. FIG. 1A is a diagram of functional elements showing the parental vector design. FIG. 1B shows HEK293 cells transformed with the parental vector. FIG. 1C HEK293 cells co-transformed with parental vector and vector expressing tamoxifen inducible ERCreER recombinase (ERCreER recombinase). Scale bar 50 μm.

FIG. 2 illustrates a puromycin resistant Sleeping Beauty transposon with ER-Cre-ER expressing cassette.

FIG. 3 illustrates a neomycin resistant PiggyBac transposon with EGFP-Neo/GW excisable TurboRFP inducible cassette.

FIG. 4 illustrates a PiggyBac transposon with a mitogen cMyc excisable RNA Dependent RNA Polymerase inducible cassette.

FIG. 5 shows flow-sort parameters for separation of viable single HEK293 cells expressing EGFP.

FIGS. 6A-6C are micrographs of HEK293 cells modified with the plasmids 2 and 3 showing bright field (FIG. 6A), EGFP fluorescence (FIG. 6B) and in overlay (FIG. 6C).

FIGS. 7A-7D are micrographs illustrating the inducible expression of HEK293 cells in bright field (FIG. 7A), sequentially modified first with plasmids 2 and 3 (FIG. 7B), followed by modification with plasmid 1 (FIG. 7C) and in overlay (FIG. 7D).

FIGS. 8A-8B show vectors designed for generation of tamoxifen inducible HEK293-ERCreER-Mitogen stable cell lines. FIG. 8A shows the modification of the parental vector containing excisable sequence of a mitogen. FIG. 8B shows the plasmid for transient expression of corresponding transposase.

FIGS. 9A-9C show the vectors designed for generation of HEK293-ERT2CreERT2-exMitogen-indGOI stable cell lines. FIG. 9A shows the modification of the parental vector with excisable mitogenic/inducible GOI cassette. FIG. 9B shows the modification of the parental vector with GOI inducible cassette. FIG. 9C shows the plasmid for transient expression of the corresponding transposase.

### DETAILED DESCRIPTION OF THE INVENTION

As used herein, the articles “a” and “an” when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the terms “comprise” and “comprising” are used in the inclusive, open sense, meaning that additional elements may be included.

As used herein, the terms “consists of” and “consisting of” are used in the exclusive, closed sense, meaning that additional elements may not be included.

As used herein, the term “includes” or “including” is used herein to mean “including, but not limited to”. The terms “includes”, “including” and “including but not limited to” are used interchangeably.

In one embodiment of the present invention, there is provided a stably-modified cell line, comprising an insertion containing an inducible recombinase and a promoter.

Further to this embodiment, the stably-modified cell line comprises a DNA cassette encoding an excisable mitogen; a DNA cassette encoding at least one inducible gene of interest (GOI); or a DNA cassette encoding the excisable mitogen and the inducible gene of interest. In this further embodiment, the at least one inducible gene of interest may encode a difficult to express protein (DTEP) or a viral particle protein. In this further embodiment, the DNA cassette encoding the excisable mitogen and the at least one inducible gene of interest may comprise a switch configured to excise the excisable mitogen and express the at least one inducible gene of interest simultaneously via the inducible recombinase.

In both embodiments, the insertion may contain a tamoxifen inducible ERCreER (ERT2CreERT2) recombinase under the control of the promoter. In both embodiments, the stably-modified cell line is a human cell line HEK293. A representative example of the stably-modified cell line is one selected from the group consisting of HEK293-ERT2CreERT2, HEK293-ERT2CreERT2-Mitogen, HEK293-ERT2CreERT2-indGOI, or HEK293-ERT2CreERT2-exMitogen/indGOI. In a preferred embodiment, the stably-modified cell line is HEK293-ERT2CreERT2-exMitogen/indGOI.

In another embodiment of the present invention, there is provided a method for producing a biological medical product, comprising inducing expression or overexpression of the gene encoding the mitogen in a population of cells comprising the stably-modified cell line described supra; increasing cell number in the population via activity of the expressed or overexpressed mitogen; inducing a recombination activity of the inducible recombinase comprising the insertion in the cells in the increased cell population; simultaneously switching off the expression or the overexpression of the mitogen and inducing expression of the at least one inducible gene of interest comprising the cells in the increased cell population to produce at least one protein or at least one viral particle protein of medical or therapeutic interest encoded by the inducible gene of interest; and isolating the protein or the viral particle protein from the cell population, thereby producing the biological medical product.

In this embodiment, the stably-modified cell line may be a human cell line. Particularly, the human cell line is HEK293. In this embodiment, the inducible recombinase may be a tamoxifen inducible ERCreER (ERT2CreERT2) recombinase. In addition, the protein may be a difficult to express protein (DTEP) or a toxic protein. Furthermore, the at least one viral particle protein may be an adeno-associated virus particle protein.

In yet another embodiment of the present invention, there is provided a scalable process for producing at least one biopharmaceutical destructive to a population of host cells stably modified to express the same, comprising adjusting at least once in the population a ratio of the host cells induced to express the at least one biopharmaceutical to the host cells in a normal mitotic cycle or an enhanced mitotic cycle.

In this embodiment, the host cells are stably modified with an excisable mitogen and at least one inducible gene of interest that expresses the biopharmaceutical, where the adjusting step comprises simultaneously switching off the expression or the overexpression of the mitogen and inducing expression of the inducible gene of interest, thereby scaling the process. In this embodiment, the host cells in the population may be HEK293 cells stably modified with an ERT2CreERT2-Mitogen-indGOI transposon. In addition, the biopharmaceutical may be a viral particle or a difficult to express protein.

In yet another embodiment of the present invention, there is provided an engineered HEK293 cell line comprising a transposon containing an inducible recombinase and a promoter and a DNA cassette encoding an excisable mitogen and an inducible gene of interest. In this embodiment, the engineered HEK293 cell line may comprise a population of HEK293-ERT2CreERT2-exMitogen/indGOI cells.

Provided herein are recombinant DNA constructs, recombinant DNA vectors or transposons, cell lines, and methods of using the same to produce engineered cell lines with enhanced mitotic activity in which gene expression can be manipulated via the activity of the DNA recombinase. The genetically engineered cell lines are suitable for the scalable industrial production of biological medical products, such as, but not limited to, toxic or difficult to express proteins (DTEP) of therapeutic value, vaccines, and gene therapy products, for example, viral-based gene therapies. The engineered cell lines also may be used for educational and research purposes.

Generally, the DNA recombinase mediated rearrangement of nuclear DNA makes it possible to conduct excision or inversion of precisely designed DNA fragments. The key feature of the invention described herein is the strategic design and positioning of DNA elements with defined properties within the DNA construct that provides control over expression of specific genes. The DNA construct defines a purposeful combination of the mentioned DNA elements that enable temporal control over cell activity. A molecular switch is created that separates cell growth (proliferation) from protein synthesis into distinct phases. Additionally, the DNA construct artificially stimulates or enhances cell division by expressing mitogen(s) that speed up cell proliferation. After the cell culture reaches optimal cell density during the cell proliferation phase, DNA recombinase mediated rearrangement or switch of the designed DNA construct halts mitogenic or proliferating activity of the cell and induce production of difficult to express protein(s).

Particularly, the engineered cell line comprises stable genetic modifications via a vector or transposon containing an inducible recombinase and one or more DNA cassettes, for example, a DNA cassette encoding a mitogen, such as an excisable mitogen and/or a DNA cassette encoding one or more specific proteins. In one example, a first DNA cassette enables induction of the expression or overexpression of an excisable mitogen gene sequence and can be switched off while a second DNA cassette enables expression of a gene of interest and can be switched on simultaneously as mitogen expression is switched off or the first DNA cassette is excised, upon induction of the recombinase. This enables the initial rapid growth of the cells via increased mitogenic activity followed by the inducible production of, but not limited to, one or more difficult to express proteins and/or toxic proteins or viral-based therapeutics as separate recombination events or enables elimination of expression of a protein or enables a combination of both inducible production and elimination of specific proteins in the same recombination event or during the same induction step. Activation of ERT2CreERT2 may lead to a single recombination or several recombinations. The number of recombination events depends on the number of inserted cassettes, i.e., insertions, flanked with Lox sites (recombination sites).

The engineered or modified cell lines may be eukaryotic cell lines, preferably mammalian cell lines, or more preferably, human cell lines. Non-limiting examples are a tamoxifen inducible cell line HEK293-ERT2CreERT2, a tamoxifen inducible HEK293-ERT2CreERT2-exMitogen cell line or a tamoxifen inducible HEK293-ERT2CreERT2-exMitogen-indGOI cell line.

Further provided by the present invention are methods and processes for bioproducing difficult to express proteins with the engineered or modified cell lines described herein. By manipulating the cell cycle and the concentration of the induction agent, it is possible to achieve a wide range of ratios between induced and uninduced cells within the treated population, ranging from low to high. While the induced population switches to production mode, the uninduced fraction of cells continues their normal or artificially enhanced mitotic cycle. Such responsiveness to manipulation in outcomes of the inducible system provided herein can be utilized to facilitate two distinct modes of bioproduction, depending on the nature of the product of interest, desired outcome, and available equipment: a) batch/fed-batch manufacturing mode, or b) steady-state continuous manufacturing in perfusion systems.

In batch/fed-batch production mode, the cell culture may be induced at least once with the addition of feed. Moreover, synchronization of the culture can be used as a separate process step or in combination with feed to achieve the most uniform and highest percentage of inducible recombination events. By manipulating the concentration of the inducible agent and the intervals between inductions in continuous perfusion mode of production, it is possible to achieve a state of cell culture in which the ratio between induced and uninduced cells provides a steady production of the product and continuous replenishment of producing cells. This mode would be highly beneficial in situations where the product is destructive to the producing cell, such as, but not limited to, the production of lytic serotypes of viral vectors for gene and cell therapies.

The following examples are given for the purpose of illustrating various embodiments of the invention and are not meant to limit the present invention in any fashion.

### Example 1

Generation of HEK293-ERT2CreERT2 Cell Lines with a Stable Expression of ERT2CreERT2 Recombinase

### Parental Vector

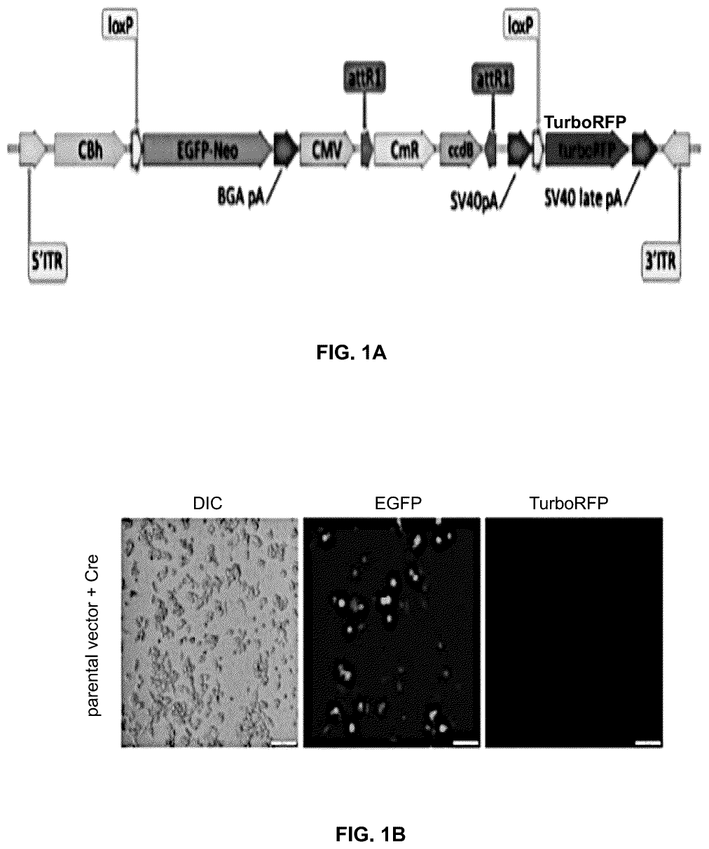

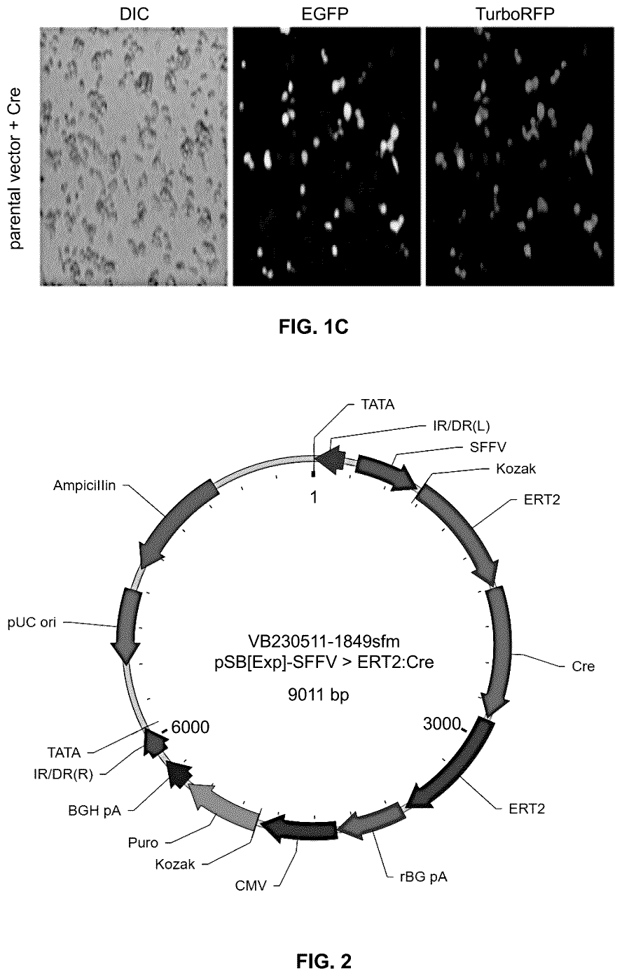

A parental vector is developed and its performance tested in an HEK293 cell line (FIG. 1A). The construct is designed to first express genes flanked by loxP cassette: (EGFP/Neo) fusion protein and any gene potentially inserted into attR1 and attR2 cloning cassette (FIG. 1B). The sequence of turboRFP is strategically positioned behind the expression cassette and is activated only by removal of the loxP cassette via Cre-mediated recombination (FIG. 1C). The parental construct may be used as a control plasmid and serves as a backbone to develop several constructs with a set of defined functional elements.

### Plasmids

DNA vectors were designed and purchased from Vectorbuilder.

A Sleeping Beauty transposon (FIG. 2, plasmid 1) contains a cassette with two functional elements: a) an ERT2CreERT2 (inducible recombinant Cre recombinase) under an SFFV promotor and b) a puromycin resistant gene under a CMV promotor. Both elements inserted in between two inverted repeats sequences (IR/DR(L/R)) that upon interaction with sleeping beauty transposase provide stable integration of the cassette. Modification of host cells (HEK293) with this plasmid makes the modified cells resistant to Puromycin. Such resistance enables antibiotic selection of stably modified cells. Additionally, ERT2CreERT2 recombinase enables inducible Cre mediated recombination of Lox flanked sequences, stably integrated into a host cell genome.

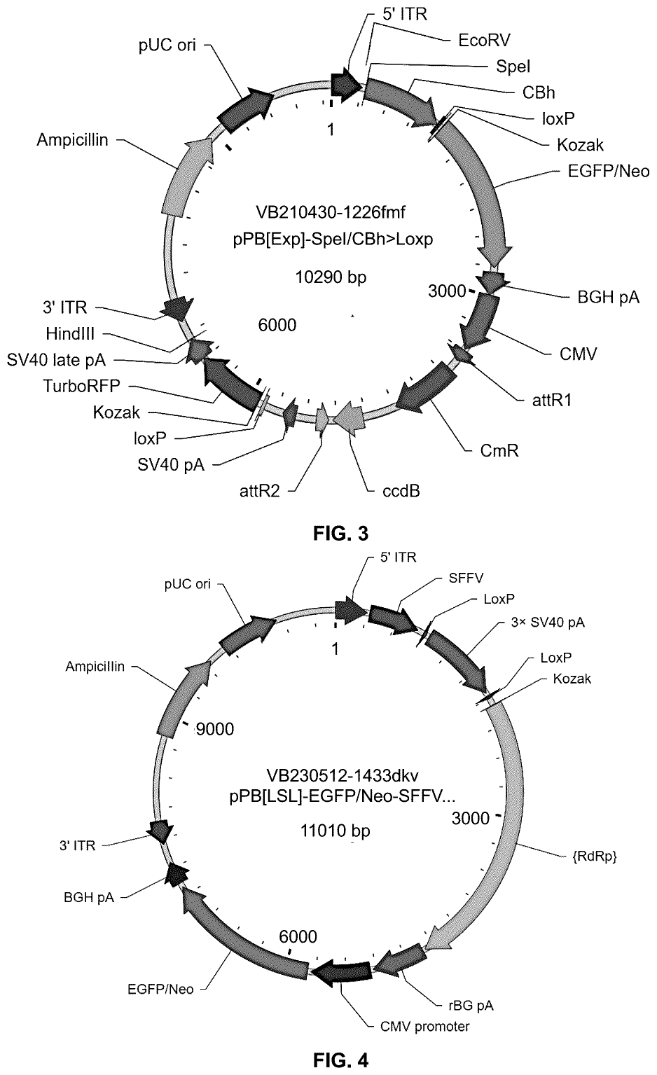

A multifunctional PiggyBac transposon (FIG. 3, plasmid 2) contains a cassette with two fluorescent proteins as selectable markers and a Gateway recombination cassette. A first fluorescent recombinant protein (EGFP/Neomycin) followed by a Gateway cloning cassette is flanked with a Lox sequence. A sequence of turbo red fluorescent protein is located immediately outside of the Lox flanked cassette. Modification of host cells (HEK293) with this plasmid makes the modified cells resistant to neomycin with a simultaneous expression of enhanced green fluorescent protein (EGFP). Such expression enables dual marker selection. The Gateway cassette serves as an acceptance cassette for cloning mitogenic or other proteins of interest. Lox elements enable the excision of EGFP/Neomycin-GateWay sub-cassette by induced ER-Cre-ER recombinase. Excision of Lox flanked sequence by inducible ERT2CreERT2 recombinase enables expression of turbo red fluorescent protein (TurboRFP). The vector may be utilized for two applications: a) as a control for functionality and efficiency of ERT2CreERT2 recombinase and b) as a plasmid for Gateway cloning and testing performance of mitogens or other proteins.

A Piggy Beck transposon (FIG. 4, plasmid 3) contains an ERT2CreERT2 excisable mitogenic cassette that expresses cMyc protein and inducible RDRP (RNA Dependent RNA Polymerase) as a protein of interest. Modification of host cells (HEK293) with this plasmid is expected to increase the rate of cell division. Upon modification of such cells with ERT2CreERT2, modified cells first divide at rates higher than unmodified cells, and express a protein of interest (RDRP) upon ERT2CreERT2 mediated recombination that switches cellular activity from enhanced proliferation to production mode.

Transformation of HEK293 Cells with Transposon-Based Inducible ERT2CreERT2 Recombinase Followed by Bulk Marker-Based Selection

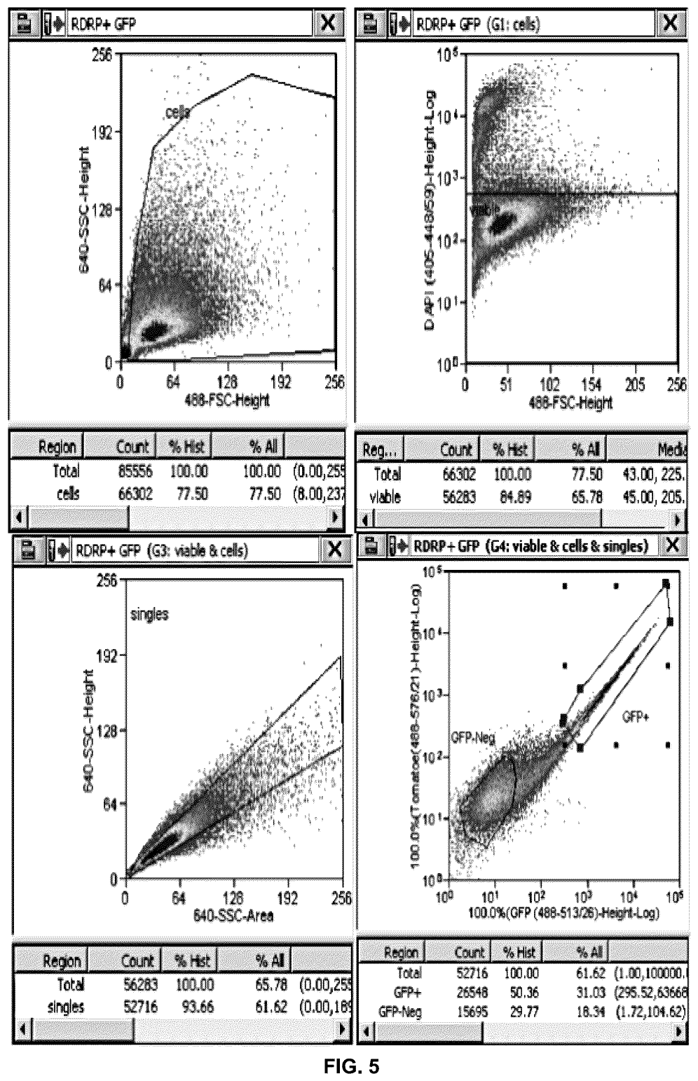

HEK293 cells were purchased from ATCC and maintained in the media recommended by ATCC. HEC 293 cells were first modified via lipofection with the plasmid 2 and plasmid 3 in combination with Piggy Beck transposase for stable integration of the constructs. Modified cells expressed EGFP and were resistant to neomycin selection. Alternatively, the modified cells may express a fluorescent protein similar to EGFP. Resistant cells expressing green fluorescent protein are expected to contain an insertion of plasmid 3. Then, secondly, the modified cells were further modified via lipofection with the plasmid 1 and subjected to puromycin selection. The culture of transformed cells is fed with an antibiotic-containing media for up to 14 days to select a bulk of resistant cells. After selection the population of modified cells were flow-sorted as a single cell into 96-well plates and subjected to clonal selection (FIG. 5).

### HEK293 Cell Clones Selection

Generally, several cell lines were generated as described. Selected cell lines demonstrate two measurable fitches: firstly, enhanced proliferation compared to the parental HEK293 cell line and secondly, tightly controlled inducible expression of the marker protein TurboRFP.

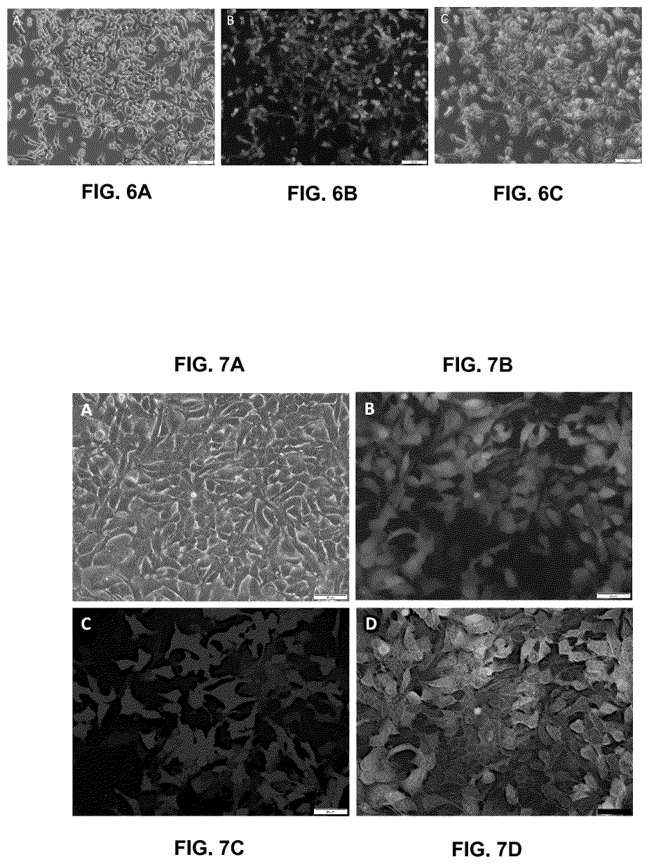

Cell clones that demonstrated enhanced proliferation by proliferation rate were selected for further expansion. All clones were expressing green fluorescent protein over multiple passages that indicates stable integration of plasmid 2 (FIGS. 6A-6C).

Cell clones were tested for presence and functionality of ERT2CreERT2 by supplementing media with 40H tamoxifen. Selected clones demonstrated inducible expression of red fluorescent protein which indicates the presence of a stably integrated ERT2CreERT2 sequence (FIGS. 7A-7D).

### Generate a Library of Stably-Modified Clones of HEK293-ERT2CreERT2 and Perform Function-Based Clonal Selection.

Once selected, the cells are distributed into 3-5 96-well plates at the density of one cell per well using single cell-selecting instruments. The plates are examined for a single cell deposition to ensure the single cell origin of potential clones. Successfully propagating clones are used to prepare clonal replica plates for the function-based clonal analysis. For this purpose, replica plates are subjected to a modification with a parental vector. The activity of Cre recombinase is estimated by scoring the level of expression of reporter turboRFP fluorescent protein upon induction of recombination. Different concentrations of inducing agent (4OH-tamoxifen) are tested. Clones with the most efficient activity of inducible recombinase are subjected to the growth curve analysis. Clones with the best combination are selected for a cell line derivation. Selected clones are consolidated on the 24- or 12-well plates and subjected for a repetitive functional analysis.

Derivation of Cell Lines from Selected Clones

A genomic characterization of the created cell lines, i.e., determine copy number and integration site of the transposon, is conducted. The cell lines selected are expanded to 3000-5000 million cells per line. The expanded cell line is cryopreserved as a master cell bank of 300-500 vials with 10 min cells/vial. Additionally, genomic DNA is extracted from representative cell lines for transposon copy number analysis and a determination of genomic position of transposon integration.

### Example 2

Generation of HEK293-ERT2CreERT2-Mitogen Cell Line(s) with a Stable Expression of Cre Recombinase Excisable Mitogenic Cassette

### Determination of the Most Efficient Mitogen or Mitogen Combinations

There are four classes of proteins (mitogens) that can stimulate cellular proliferation: 1) extracellular growth factors; 2) transmembrane signaling proteins; 3) cytoplasmic cellular mitogens; and 4) nuclear transcription factors. A panel of four classes of mitogenic protein sequences are collected and inserted into an expression vector and tested for their ability to enhance a cell division upon transient transformation of HEK293 cells. Activity of separate mitogens or their combinations is assessed by a growth curve analysis of the transformed cells. The mitogen(s) with the highest ability to stimulate cell division are cloned into the excisable cassette of the parental transposon vector. At minimum, four different constructs are generated and verified by the restriction map analysis. Successful clones are subjected to an endotoxin-free Maxiprep plasmid preparation.

Transformation of HEK293-ERT2CreERT2 Cell Line(s) with Transposon-Based Excisable Mitogenic Cassette

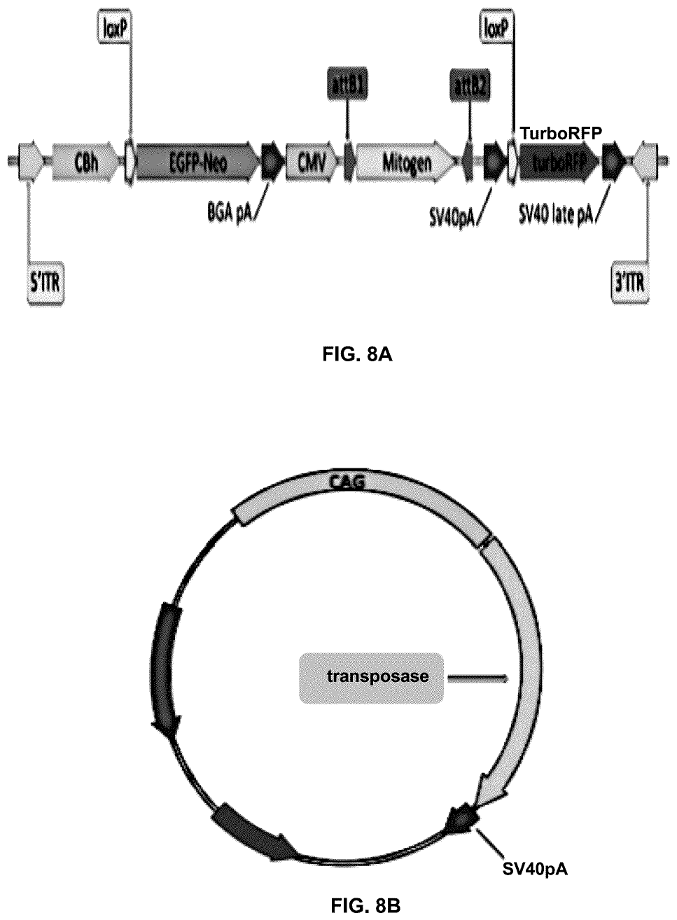

HEK293-ERT2CreERT2 cells are transformed to HEK293-ERT2CreERT2-Mitogen cell lines with a cocktail containing transposon with Cre excisable mitogenic cassette (FIG. 8A) intended for a stable integration into cellular genome and a plasmid containing transposase and Neomycin resistant genes (FIG. 8B) intended to transiently provide transposase for the corresponding transposon integration. The culture of the transformed cells is fed with an antibiotic-containing media for up to 14 days to select a bulk of resistant cells. Alternatively, or additionally, a cell-sorting may be deployed to select cells expressing EGFP.

A library of stably-modified tamoxifen-inducible HEK293-ERT2CreERT2-Mitogen clones are generated as described in Example 1. Clones with the best combination of proliferating and recombination activities are selected for the cell line derivation. The selected clones will be consolidated on 24- or 12-well plates and subjected for repetitive functional analysis and further expansion. Cell lines are derived from selected clones, genomic characterization of the created cell lines is performed and a Master Cell Bank is generated as in Example 1.

### Example 3

HEK293 Cell Lines Generated Using HEK293-ERT2CreERT2 and/or HEK293-ERT2CreERT2-Mitogen Cell Lines for Production of Viral Particles or Other Difficult to Produce Proteins

### Development and Verification of Transposon-Based DNA Cassette for Stable Integration

An agenetic construct may be generated in which recombinase mediated excision (removal) of the actively transcribed cassette activates transcription of the second cassette so that only one cassette is transcribed at a time. Such an arrangement of inducible recombinase excisable mitogenic/inducible gene of interest (GOI) cassettes, or only an inducible gene of interest cassette is useful for the production of AAV2-GFP viral particles or selected difficult to produce proteins of interest. Lines of modified mitogenic parental vectors are developed for inducible expression of the gene of interest: a) AAV helper genes; b) AAV rep and cap genes; and c) difficult to produce proteins. The specific gene of interest sequence is cloned into the inducible cassette of the parental transposon vector.

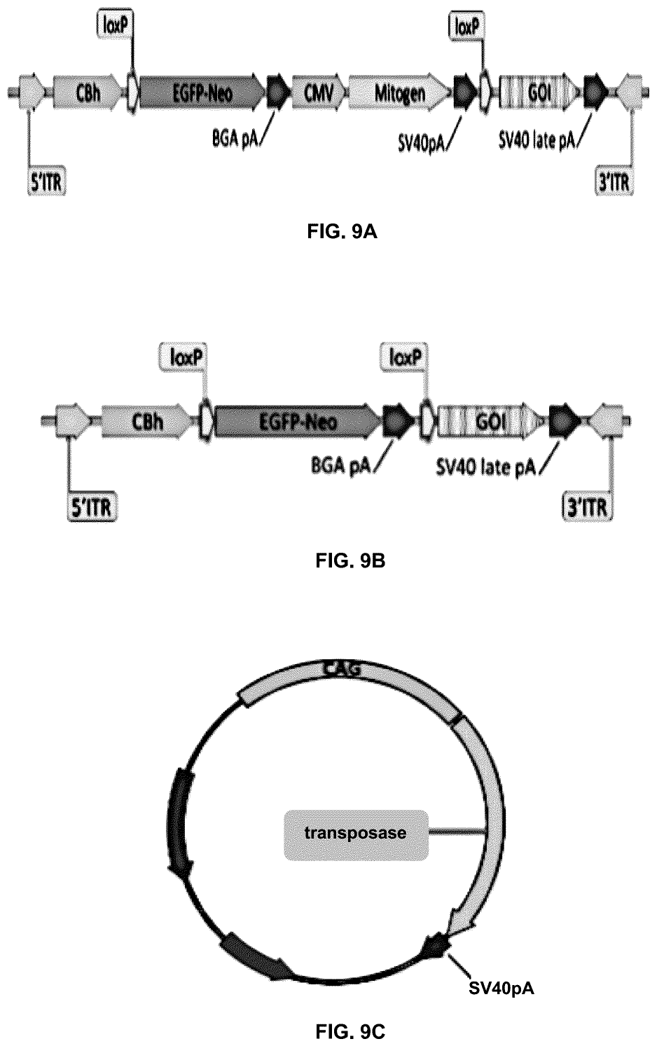

There are two parallel approaches. First is the development of mitogen-excisable/GOI-inducible cassette for a modification of the tamoxifen inducible HEK293-ERT2CreERT2 cell line (FIG. 9A). Second is the development of a gene of interest-inducible cassette for a modification of the tamoxifen inducible HEK293-ERT2CreERT2-Mitogen cell lines (FIG. 9B). The first approach results in the generation of a cell line which is capable of an excision of a mitogenic cassette that leads to an activation of expression of the gene of interest in the same recombination event upon activation or induction of the ERT2CreERT2 with 4OH-tamoxifen. The second approach results in the generation of a cell line which is capable of excision of a mitogenic cassette and activation of an expression of gene of interest as two separate recombination events during a single stimulation of the ERT2CreERT2 with the 4OH-tamoxifen.

Transformation of HEK293-ERT2CreERT2 with a Transposon-Based Excisable Mitogenic/Inducible GOI Cassette and HEK293-ERCreER-Mitogen Cell Line(s) with a Transposon-Based Inducible GOI Cassette for Production of Viral Particles or Difficult to Produce Proteins

HEK293-ERT2CreERT2 and/or tamoxifen inducible HEK293-ERT2CreERT2-Mitogen cell lines are transformed with a cocktail containing corresponding transposon with a Cre excisable/inducible GOI cassette (FIGS. 9A-9B) intended for a stable integration into cellular genome and a plasmid containing a transposase (FIG. 9C) intended to transiently provide transposase for a corresponding transposon integration into a host genome. The culture of transformed cells is fed with an antibiotic-containing media for up to 14 days in order to select a bulk of resistant cells. Alternatively, or additionally, cell sorting may be deployed to select cells expressing fluorescent marker proteins.

Once selected a library of stably-modified HEK293-ERT2CreERT2-exMitogen/indGOI clones are generated as described in Example 1. Successfully-propagating clones are used to prepare clonal replica plates for the function-based clonal analysis. For this purpose, replica plates are analyzed for product yield and quality. Clones with the highest proliferation activity and best productivity are selected for further propagation and cell line derivation. Selected clones are consolidated on 24- or 12-well plates and subjected for repetitive functional analysis and further characterization. Cell lines are derived from selected clones, genomic characterization of the created cell lines is performed and a Master Cell Bank is generated as in Example 1.

### REFERENCES

Chahal et al. Journal of Virological Methods 196:163-173, 2014.
Dobrowsky et al. Current Opinion in Biomedical Engineering 20, 2021.
El Andari, J. and Grimm, D. Biotechnology Journal 16, 2021
Formas-Oliveira et al. Biotechnology Journal 15, 2020.
Grieger et al. Molecular Therapy 24:287-297, 2016.
Jaluria et al. BMC Biotechnol 7:71, 2007.
Mori et al. Scientific Reports 10, 2020.
Pulix et al. Current Opinion in Biotechnology 71:18-24, 2021.
Syvyk, A. E. and Syvyk, T. L. Exp Oncol 41:95-105, 2019.
Syvyk, T. and Syvyk, A. Journal of Microbiology Biotechnology and Food Sciences 7:519-523, 2018.
Tan et al. Frontiers in Bioengineering and Biotechnology 9, 2021.
Thorne et al. Human gene therapy 20:707-714, 2009.
Wang et al. Nature Reviews Drug Discovery 18:358-378, 2019.
Wright, J. F. Gene Therapy 15:840-848, 2008.
Zhang et al. Biotechnol Bioeng 114:2539-2549, 2017.

What is claimed is:

1. A stably-modified cell line, comprising:
an insertion containing an inducible recombinase and a promoter.

2. The stably-modified cell line of claim 1, wherein the insertion contains a tamoxifen inducible ERCreER (ERT2CreERT2) recombinase under the control of the promoter.

3. The stably-modified cell line of claim 1, further comprising:
a DNA cassette encoding an excisable mitogen;
a DNA cassette encoding at least one inducible gene of interest; or
a DNA cassette encoding the excisable mitogen and the at least one inducible gene of interest.

4. The stably-modified cell line of claim 3, wherein the inducible gene of interest encodes a difficult to express protein or a viral particle protein.

5. The stably-modified cell line of claim 3, wherein the DNA cassette encoding the excisable mitogen and the at least one inducible gene of interest comprises a switch configured to excise the excisable mitogen and express the at least one inducible gene of interest simultaneously via the inducible recombinase.

6. The stably-modified cell line of claim 1, wherein said cell line is a human cell line HEK293.

7. The stably-modified cell line of claim 6, wherein the cell line is selected from the group consisting of HEK293-ERT2CreERT2, HEK293-ERT2CreERT2-Mitogen, HEK293-ERT2CreERT2-indGOI, or HEK293-ERT2CreERT2-exMitogen/indGOI.

8. The stably-modified cell line of claim 7, wherein the cell line is HEK293-ERT2CreERT2-exMitogen/indGOI.

9. A method for producing a biological medical product, comprising:
inducing expression or overexpression of the gene encoding the mitogen in a population of cells comprising the stably-modified cell line of claim 8;
increasing cell number in the population via activity of the expressed or overexpressed mitogen;
inducing a recombination activity of the inducible recombinase comprising the insertion in the cells in the increased cell population;
simultaneously switching off the expression or the overexpression of the mitogen and inducing expression of the at least one inducible gene of interest comprising the cells in the increased cell population to produce at least one protein or at least one viral particle protein of medical or therapeutic interest encoded by the at least one inducible gene of interest; and
isolating the protein or the viral particle protein from the cell population, thereby producing the biological medical product.

10. The method of claim 9, wherein the stably-modified cell line is a human cell line.

11. The method of claim 10, wherein the human cell line is HEK293.

12. The method of claim 9, wherein the inducible recombinase is a tamoxifen inducible ERCreER (ERT2CreERT2) recombinase.

13. The method of claim 9, wherein the protein is a difficult to express protein or a toxic protein.

14. The method of claim 9, wherein the at least one viral particle protein is an adeno-associated virus particle protein.

15. A scalable process for producing at least one biopharmaceutical destructive to a population of host cells stably modified to express the same, comprising:
adjusting at least once in the population a ratio of the host cells induced to express the at least one biopharmaceutical to the host cells in a normal mitotic cycle or an enhanced mitotic cycle.

16. The scalable process of claim 15, wherein the host cells are stably modified with an excisable mitogen and at least one inducible gene of interest that expresses the biopharmaceutical, said adjusting step comprising simultaneously switching off the expression or the overexpression of the mitogen and inducing expression of the at least one inducible gene of interest, thereby scaling the process.

17. The scalable process of claim 15, wherein the host cells in the population are HEK293 cells stably modified with an ERT2CreERT2-Mitogen-indGOI transposon.

18. The scalable process of claim 15, wherein the biopharmaceutical is a viral particle or a difficult to express protein.

19. An engineered HEK293 cell line comprising a transposon containing an inducible recombinase and a promoter and a DNA cassette encoding an excisable mitogen and an inducible gene of interest.

20. The engineered HEK293 cell line of claim 19, comprising a population of HEK293-ERT2CreERT2-exMitogen/indGOI cells.
